(19) United States
(12) Patent Application Publication
(10) Pub. No.: US 2010/0036300 A1
(43) Pub. Date: Feb. 11, 2010

(54) Orthopaedic Devices

(76) Inventors: David Sheehan, County Waterford (IE); Linda Ryan, Dublin (IE)

Correspondence Address: JACOBSON HOLMAN PLLC, 400 SEVENTH STREET N.W., SUITE 600, WASHINGTON, DC, 20004, US

(21) Appl. No.: 12/311,495

(22) PCT Filed: Oct. 3, 2007
(86) PCT No.: PCT/IE2007/000091

§ 371 Date: Apr. 1, 2009

(30) Foreign Application Priority Data
Oct. 3, 2006 (IE) 2006/0719
Oct. 3, 2006 (IE) 2006/0720
Oct. 3, 2006 (IE) 2006/0721
Oct. 3, 2006 (IE) 2006/0722
Mar. 9, 2007 (IE) 2007/0155
Mar. 9, 2007 (IE) 2007/0156
Mar. 9, 2007 (IE) 2007/0159
Mar. 9, 2007 (IE) 2007/0160
Aug. 29, 2007 (IE) S2007/0620

(51) Int. Cl. A61F 5/00

(52) U.S. Cl. 602/7

(57) ABSTRACT

A custom mouldable orthopaedic device (1) adapted, in use, to be volumetrically variable, includes a first member (2), adapted to be conformable around a body part comprising a sheet of thermoplastic material e.g. a polycaprolactone polymer, having first (3) and second (4) opposed edge regions and a second member (21) comprising a layer of an extensible material, e.g. a hydrophilic spacer fabric, bonded to one surface of the first member and adapted to form an interposing layer between the first member and the body part when said first member is adapted to conform around said body part, wherein at least a portion of at least one of said edge regions has a thickness less than that of the region (5) intermediate said edge region.

This invention relates to devices for splinting, bracing or supporting parts of animal bodies. Particularly this invention relates to devices for supporting parts of the human body, e.g. a wrist or arm splint, otherwise known as “orthopaedic devices”.

Orthopaedic device is a term that is used to describe medical structures such as casts, splints, supports, braces and other means utilized to support, immobilize restrain, protect and position body portions. They are used in many fields, including the physical medicine and rehabilitation fields, general medicine, neurological field, and the veterinary field. They are also used to prevent recurrence of previous disabilities, and to prevent discomfiture and subsequent disability.

Immobilisation of fractured or injured limbs is the process of restraining a joint or limb in place with a splint, cast, or brace. This is done to prevent the injured area from moving or being disturbed during the healing process.

Hitherto, the body portion is splinted by wrapping a resin or plaster-of-Paris impregnated bandage or tape which is then allowed to “set” to form a tubular supporting splint. Splints of this type are removed by cutting or sawing the rigid splint followed by breaking open of the structure. Splints of this type are usually removed only under medical supervision.

In our prior application WO2006027763 there is described a conformable splinting device inter alia comprising a sheet of thermoplastic material bonded to a skin contacting fabric layer wherein the thermoplastic sheet is configured to be both flexible and extensible when heated to a softening temperature and wherein the skin contacting layer is adapted to conform to the configuration of the thermoplastic sheet when applied around a body part. Splinting devices of this type may be further adapted to permit ready removal from around the body portion, without the need for mechanical destruction of the device.

One disadvantage of this type of splint, in common with conventional splints, is that they are not readily adjustable to accommodate volumetric changes in the splinted body portion during the healing process. The devices disclosed in WO2006027763 have a preformed shapes designed to encase a specific body portion, such as the wrist. However, it is usually necessary to produce each shape in different sizes to accommodate the size of the body portion under treatment.

This invention seeks to alleviate some of the problems of prior art splinting devices by the provision of a volumetrically variable orthopaedic device which may custom moulded to suit an individual body portion.

Thus, In accordance with the present invention there is provided a custom mouldable orthopaedic device adapted, in use, to be volumetrically variable, including a first member, adapted to be conformable around a body part comprising a sheet of thermoplastic material having first and second opposed edge regions and a second member comprising a layer of an extensible material, bonded to one surface of the first member and adapted to form an interposing layer between the first member and the body part when said first member is adapted to conform around said body part, wherein at least a portion of at least one of said edge regions has a thickness less than that of the region intermediate said edge region.

Thus such devices, which conform to the shape of the body part may be easily removed and reused.

The present invention discloses a single cast designed to function both as a primary and as a secondary treatment for limb fractures and injuries such as the distal forearm area. Trimmable sections at a variety of locations are present to make the cast adjustable to accommodate main population sizes. The trimmable sections are designed such that there are no sharp edges in direct contact with the limb. It is difficult to develop a device for treating limb injuries that will fit the entire population based on valid anthropometric data using a single sized product. By the incorporation of trimmable sections and volumetric adjustability in the present invention, and utilising the stretchability of the cast material and padding material, it is possible to create a “one-size-fits all” device.

The devices of the invention may be used for both primary treatment stage, for example for the first 2 to 3 days following a fracture of an arm bone, and for the secondary treatment stage, for example for the following 2 to 3 weeks. During the primary treatment stage a certain degree of swelling may occur. During the secondary treatment stage, the swelling will probably be reduced but it will still be important for the arm to be securely supported. Therefore it is desirable for the splint member to have a greater interior volume during the primary treatment stage than during the secondary treatment stage.

In order to accommodate the volume reduction without increasing the bulkiness of the device, the thickness of the second edge region may be less than the thickness of the first in order to minimise the overall thickness of the splint member at the overlap. The intermediate and first edge regions may have the same thickness. Alternatively both edge regions may be thinner than the intermediate region such that, for example, when the edge regions are overlapped their combined thickness is substantially the same thickness as the intermediate region. The change in thickness between the intermediate region and an edge region may be gradual or stepwise

According to an embodiment of the invention, the region of the sheet intermediate to the opposed edges may define two parts, the first part may be movable relative to the second part. This embodiment provides for ease of removal of the splint member from a patient. Aptly, a hinge component is provided, linking the first and second parts. The hinge component may be formed integrally with the first and second parts of the first member as one or more thinned sections of the first member formed between the first and second parts. Alternatively the hinge component may comprise one or more separate, spaced apart external hinges secured to the first and second parts. The hinge component should lie on an axis generally parallel to the longitudinal axis of the orthopaedic device. A strip element made from a durable material such as nylon, can be fixed over the axis of the hinge. This acts as a backup hinge to counteract possible breaks in the underlying hinge element that could occur due to polymer fatigue when the device is opened and closed frequently as in rehab applications.

The surface configurations on each side of the sheet are preferably similar and flat such that the splint member may be used to splint either the right or the left body portion.

According to a second embodiment of the invention the extensible body contacting layer may consist of an elastically extensible material. Preferably the elastically extensible material has a low elastic recovery, to ensure that the spacer member does not inhibit adjustment of the size/volume/position of the splint member. Aptly, the properties of the elastically extensible material are such that the force required to extend a 100 mm×30 mm×2.3 mm sample of the material by 40% of its original length is not more than 2.5N, more preferably not more than 1.2N. The measurements for the low elastic recovery of the padding are for a specific fabric that is used for the splint device.

Preferably, the body-contacting layer is fabricated from a class of materials known in the art as spacer fabrics

Suitable materials for the body contacting layer include woven, non-woven, knitted or foam materials.

The orthopaedic devices of the invention may thus be provided with padding material attached to it, such that there is no need for any further padding material in contact with the skin. Not only should the padding material be stretchable, and skin-contact compatible, desirably it should also be breathable to allow normal skin transpiration

In one embodiment the invention may provide a padding material which itself is hydrophilic or has a hydrophilic layer with wicking properties. The wicking properties of this padding material can reduce the occurrence of skin maceration. The padding material may have specific material properties in which the wicking properties reduce the moisture contact on the skin. This may be achieved by capillary action or by means of a hydrophilic coating on the padding. Preferably the padding layer is breathable and aptly will have a moisture vapour permeability of at least 300 gm/m²/24 hours.

In order to ensure adequate breathability of the devices of the present invention as a whole, preferably the first member will have a plurality of openings extending therethrough, thus defining a web-like configuration. More preferably substantially all of the first member will be provided with such openings at least in the region intermediate one or both edge regions.

The orthopaedic devices of the present invention are readily removable. This property of ready removal and re-use whilst being of benefit to both the medical practitioner and patient may result in problems of a different nature.

When the medical practitioner first applies the splint in its flexible and extensible state, the damaged body portion is usually manipulated to ensure that the splint “sets” in a configuration which will properly support the body portion. Due to its construction the patient will be able to readily remove the splint without medical supervision, for example because it becomes uncomfortable or wet. Such an unsupervised action may have unfortunate consequences both medical for the patient and legal for the medical practitioner if the body portion is incorrectly supported during the healing process.

The present invention seeks to address this problem by providing means for
Deterring any unauthorised removal of such splints by means of a sealed splint.
Detecting any unauthorised removal and replacement of such splints by means of a visual indicator.

Thus in accordance with a further embodiment of the invention there is further provided an orthopaedic device characterised in that at least a portion of the layer of extensible material extends beyond said first edge and in that a third member comprising a tab is flexibly attached to the extended portion of the extensible material layer and wherein said tab comprises a material which is adapted to be permanently bonded to said first member in the region of the second edge when said device is conformed around the body portion.

Typically, the thermoplastic material forming the first member is a low melting polymer such as a polycaprolactone optionally formulated with a ligno-cellulose derivative such as those describe in WO2006027763. Such materials are sufficiently rigid at ambient temperature to support body parts such as limbs. Preferably, the tab material is malleable and consists of the same material as that used for the first member

The extensible material, at least in the region to which the tab is flexibly attached, will be an elastic material. This will enable the third member to extend without rupturing to accommodate any dimensional changes in the device.

In one embodiment, the invention may relate to orthopaedic materials and methods for treating primary and secondary lower forearm injuries or other limb injuries, such as distal leg fractures or humeral injuries. In one embodiment the invention may provide improved methods for immobilisation, bracing, casting, protection and support of limbs and body parts of humans and animals. Examples of the field of use of this invention include, but are not limited to, the treatment of distal radius or ulna fractures, scaphoid fractures, metacarpal and other kind of fractures or injury such as soft tissue injuries, and can be used for primary and secondary casting.

In one embodiment the invention may provide a volumetrically adjustable splint which may include the features of:
specific trimmable sections;
a half moon area;
a roll back area around the thumb;
hinge sections along the cast;
a thinned section along the side of the cast;
stretchable and trimmable sections;
hinged sections along the cast wherein there exists a tab;
a bony prominence recess pressure relief area;
a volumetric adjustable tab;
slots/grooves positioned to facilitate positioning of tightening/retention straps;
a strap having a buckle which may include a D-loop section which has a positional feature to fit into the slots;
the cast material may be of a thermoplastic which contains specific additives that may change the thermal characteristics of the material such that improved activation times may result;
fragrances added;
A hydrophilic lining with wicking properties.

The invention will be more clearly understood from the following description of some embodiments thereof, given by way of example only, with reference to the accompanying drawings, in which:

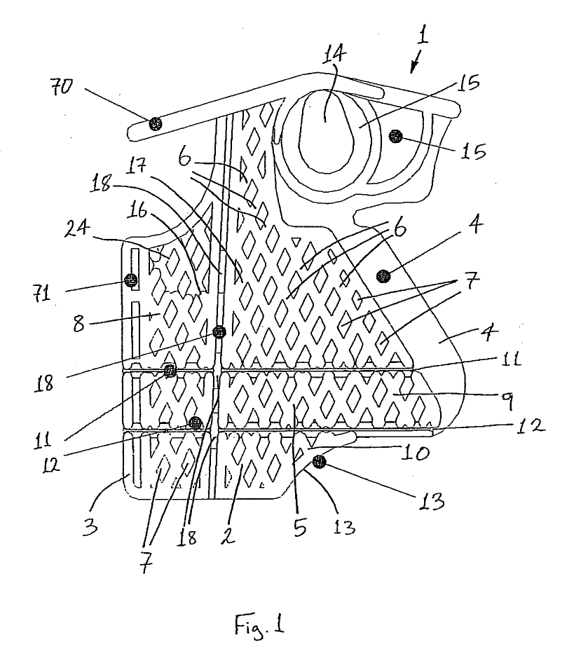

FIG. 1 is a plan view of a splint device according to the invention;

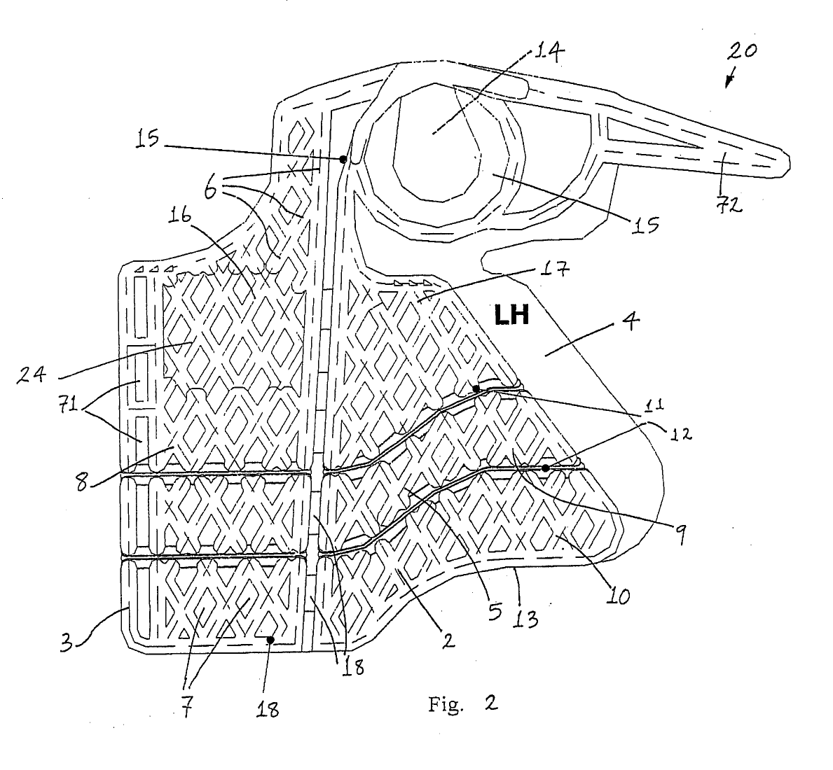

FIG. 2 shows a plan view of the unformed cast of the invention. The roll back area, trimmable sections and the hinge section are shown.

FIGS. 2 to 5 are plan views of another splint device according to the invention;

FIG. 3 shows a plan view of the unformed cast of the invention. The tamper-proof tab, the volumetrically adjustable tab for the hand and the half moon areas are shown.

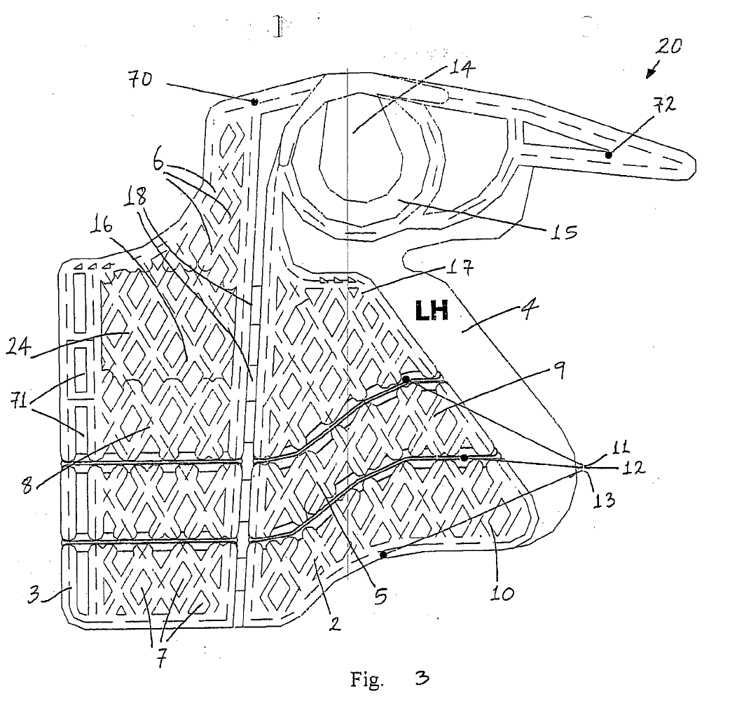

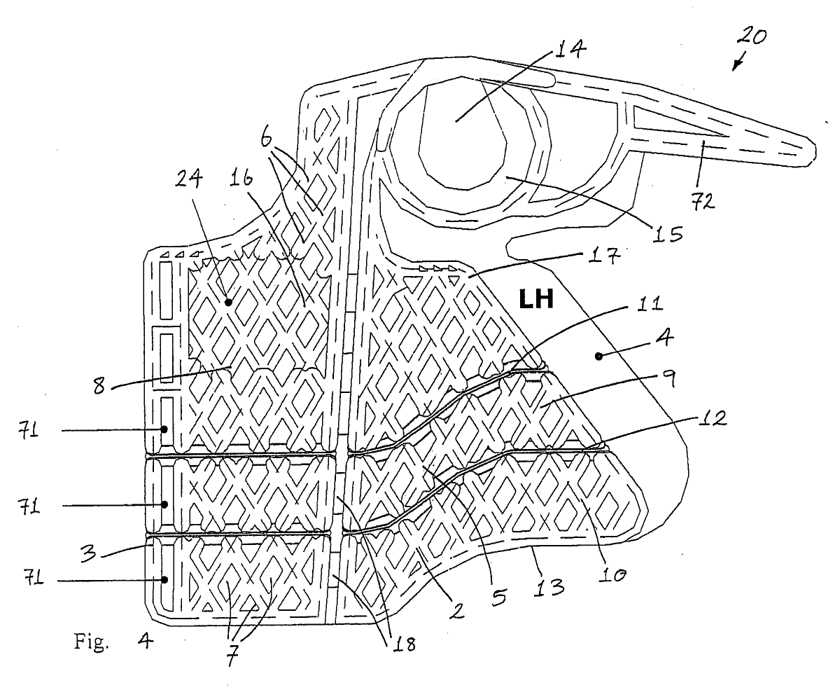

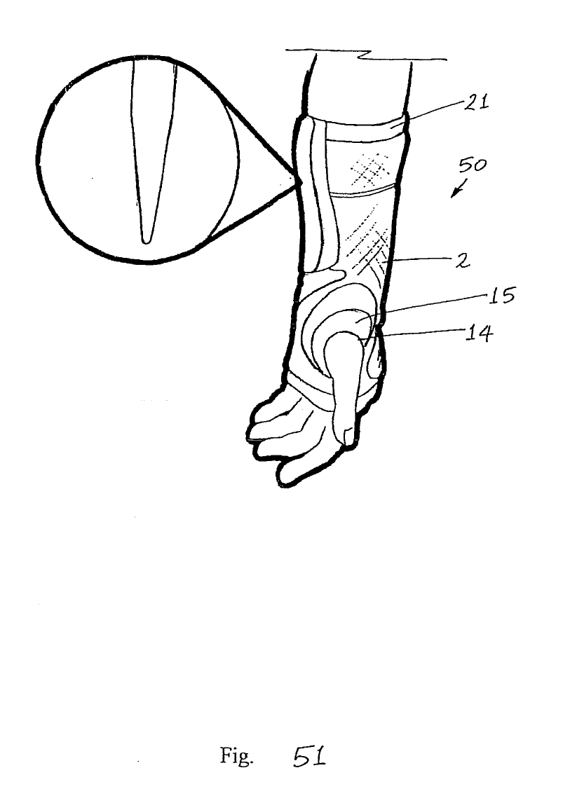

FIG. 4 details slots to facilitate positioning of straps or retaining devices to aid the volumetric adjustability of the invention. These are also areas for the strap buckle, such as a D-loop with a lug as shown in FIGS. 27 and 50 to locate such that any lateral movement of the strapping mechanism is restricted. The volumetric adjustability of the invention offers primary treatment of injuries or fractures with adjustment for increases in swelling. The volumetric adjustability of the invention offers secondary treatment of the injuries or fractures of limbs by enabling adjustment for reduction in swelling. This volumetric adjustability also allows for adjustment in the case of conditions such as muscular atrophy. The bony prominence area, for example an ulna recess area and the thinned section along the side of the cast are shown in the plan view of the invention.

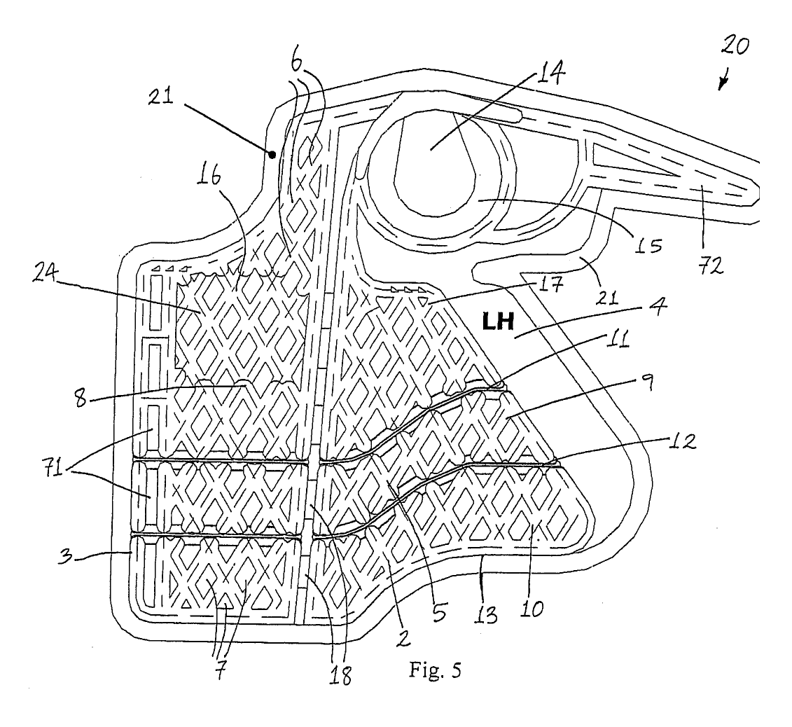

FIG. 5 shows a plan of the padding adhered to the cast material. This padding acts as a support for the activated pliable cast material during application. It also acts as a thermal insulator between the skin and the heated activated cast.

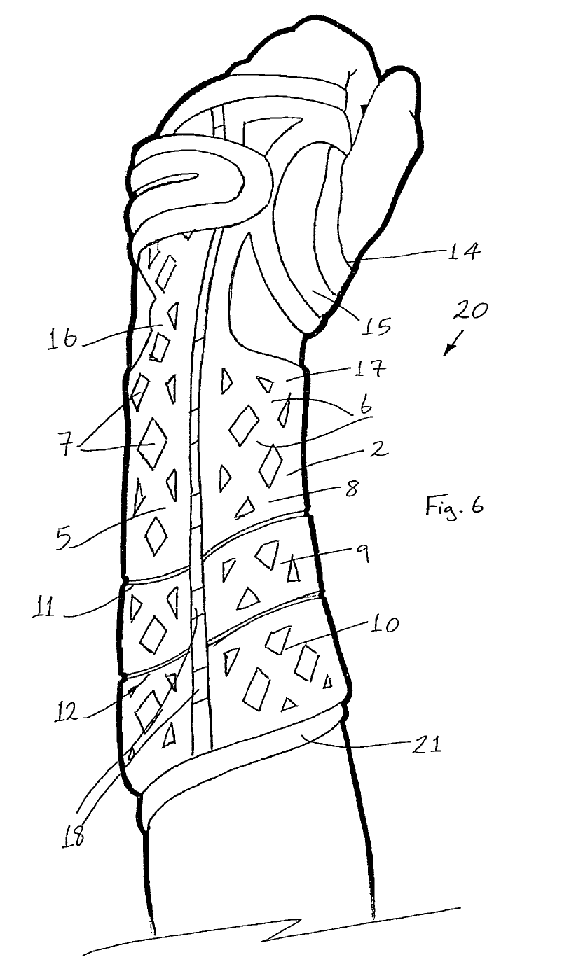

FIG. 6 shows the formed device on an injured or fractured arm.

FIGS. 7 to 9 are isometric views of the splint device of FIGS. 2 to 5, in use.

FIGS. 6 to 8 illustrate the Thermoplastic Universal mouldable device with volumetric adjustability formed around the distal forearm of either the right hand side (RHS) or the left hand side (LHS).

FIG. 10 illustrates the trimmed detail and is a plan view of a part of the splint device of FIGS. 2 to 5;

FIG. 11 is a view along line A-A in FIG. 10;

FIG. 12 is an enlarged view of a part of the splint device of FIG. 11;

FIG. 13 illustrates the thinned section showing the tapered region and is a view similar to FIG. 12 of another splint device according to the invention;

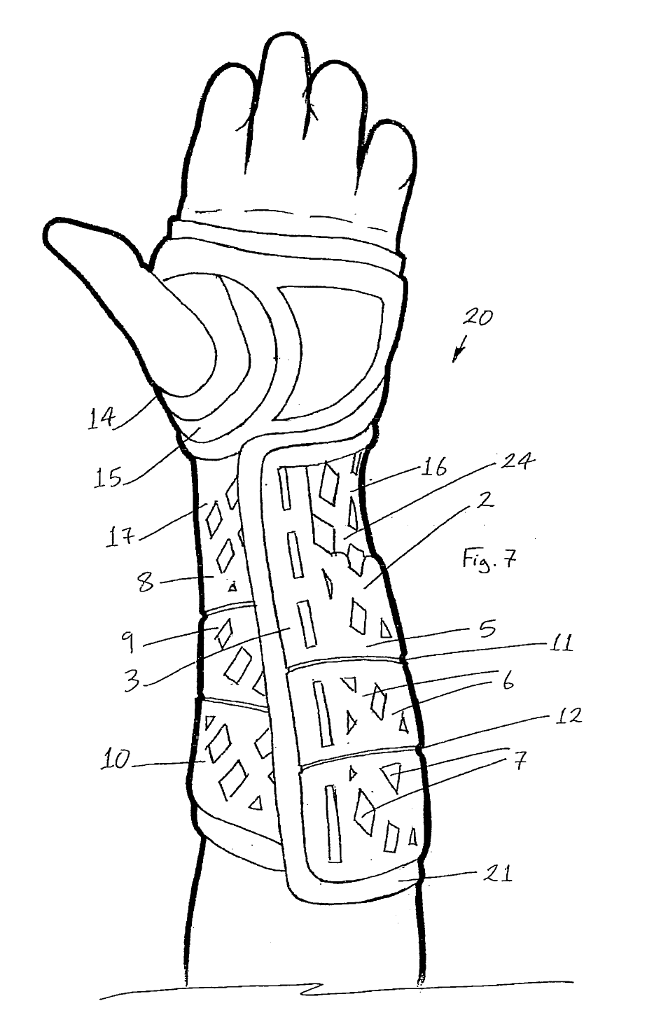

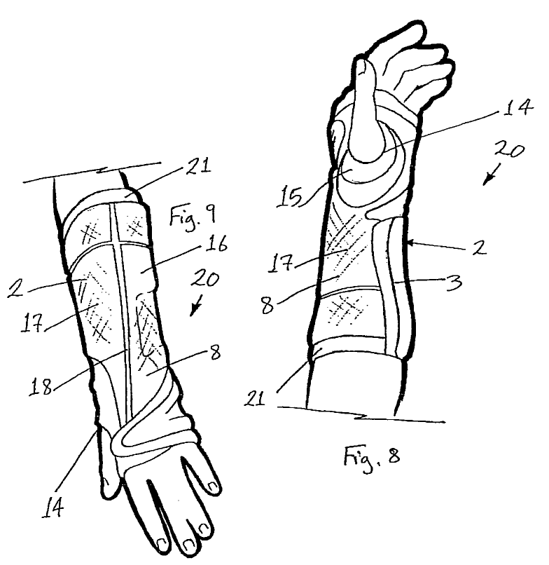

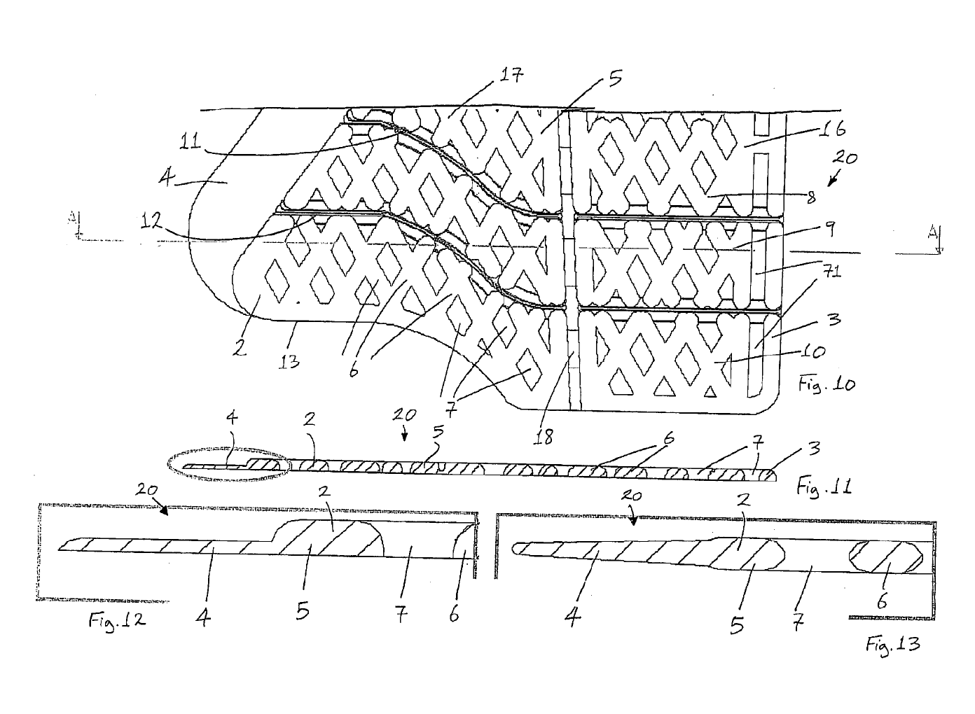

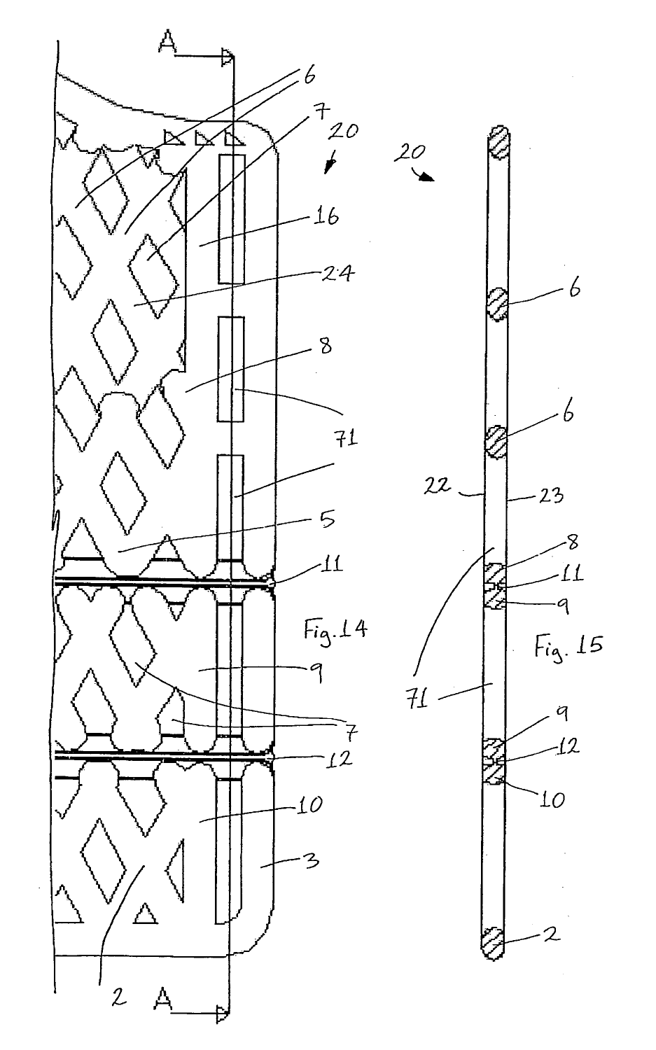

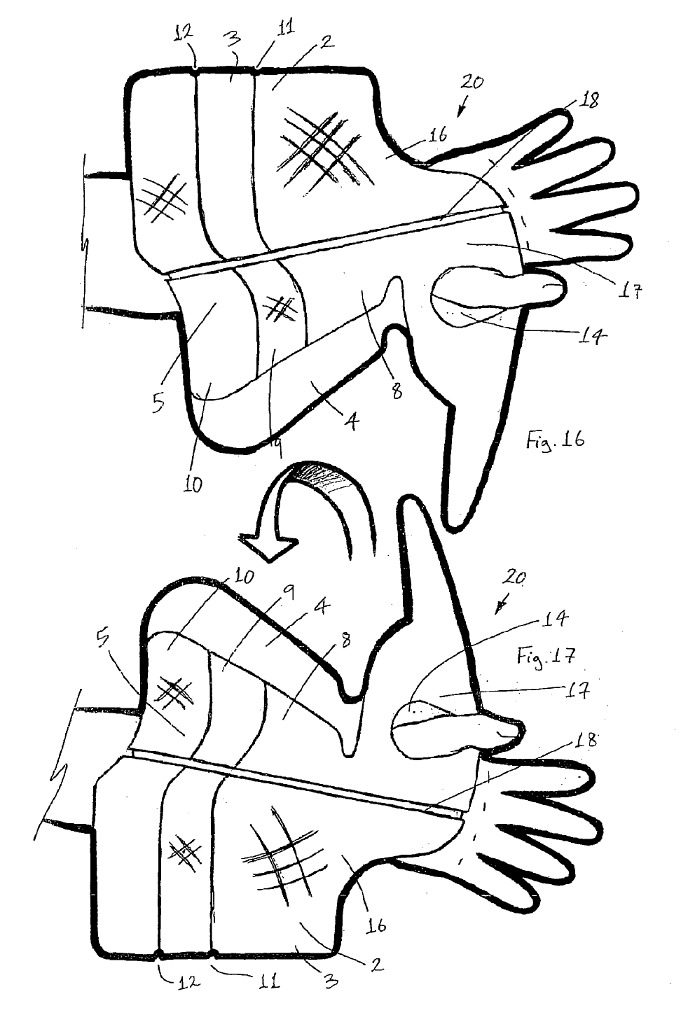

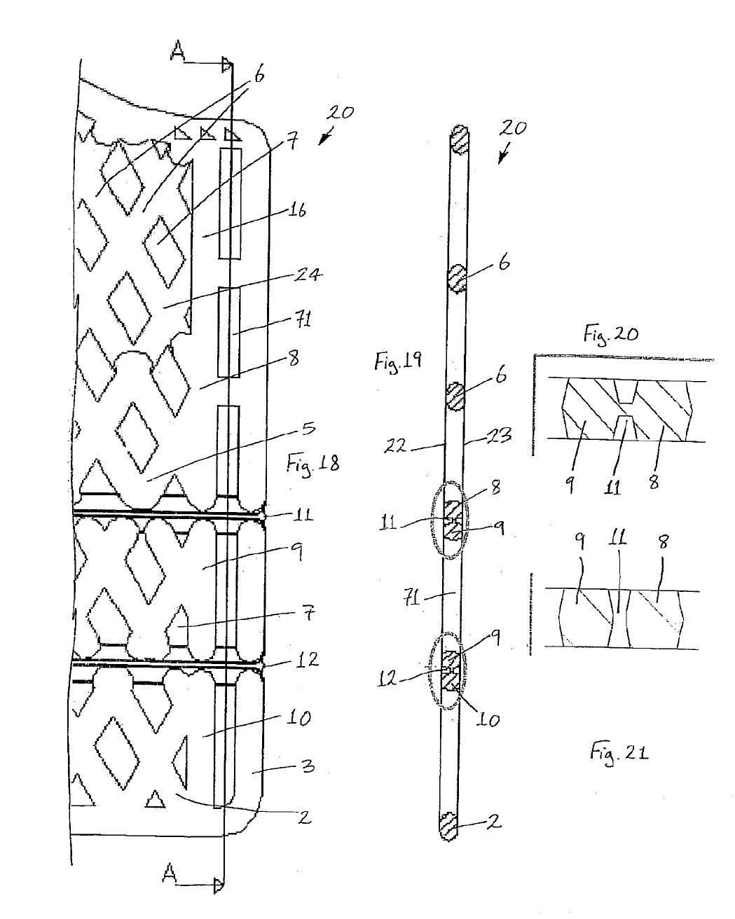

FIG. 14 is a plan view of a part of the splint device of FIGS. 2 to 5.

FIG. 15 illustrates the section of cast showing flat top and bottom and is a view along line A-A in FIG. 14;

FIGS. 16 and 17 are plan views of the unformed splint device of FIGS. 2 to 5, sifting on either the right or the left hand. The universal applicability of the device is clearly demonstrated.

FIG. 18 is a plan view of a part of the splint device of FIGS. 2 to 5;

FIGS. 18 to 21 show the trimmable sections located at specific areas of the device. These trimmable sections can tailor any size device to fit comfortably to most of the population. The trimmable section is located in such a manner that the trimmed edges are in a recess and thus will not come in direct contact with the skin.

FIG. 19 is a view along line A-A in FIG. 18;

FIGS. 20 and 21 are enlarged views of a part of the splint device of FIG. 19;

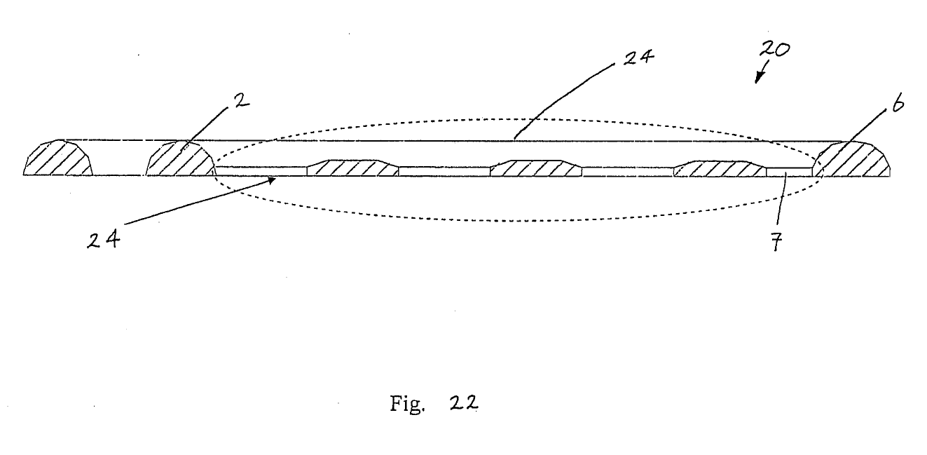

FIG. 22 is a cross-sectional, side view of a part of the splint device of FIGS. 2 to 5;

FIG. 22 shows a cross-section of the bony-prominence area referenced in FIG. 4.

FIGS. 23 and 24 are isometric views of another splint device according to the invention, in use;

FIGS. 23 to 26 show details of a hinged section along the length or part of the length of the device. The number of hinged sections can range from 1 to 6.

FIGS. 25 and 26 are isometric views of a further splint device according to the invention, in use;

FIG. 27 is an isometric view of a part of an urging means of the splint device of FIGS. 2 to 5;

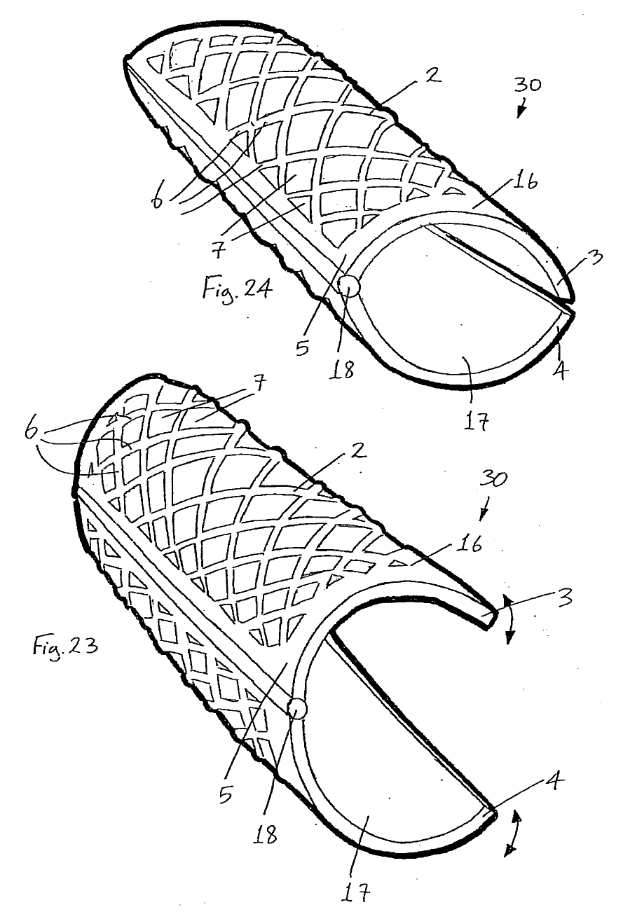

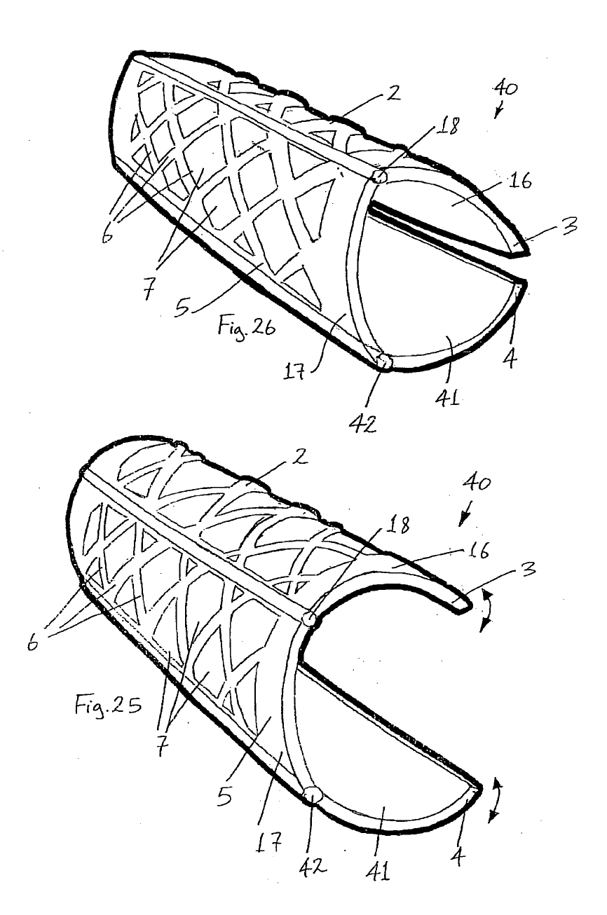

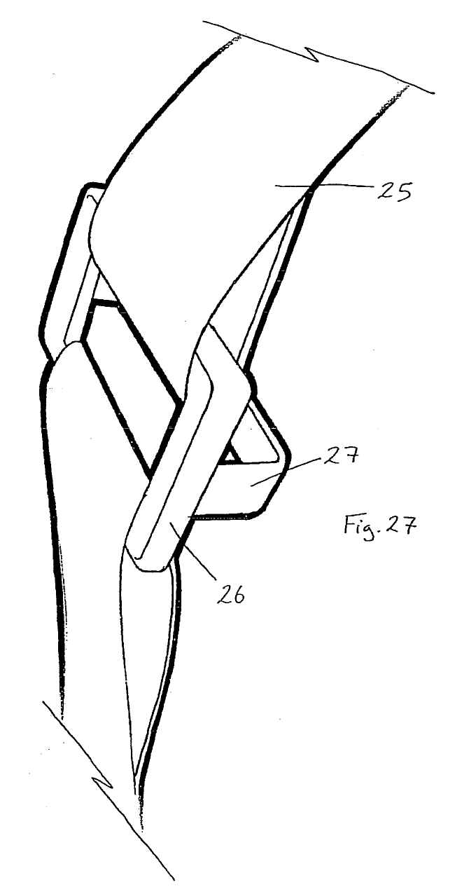

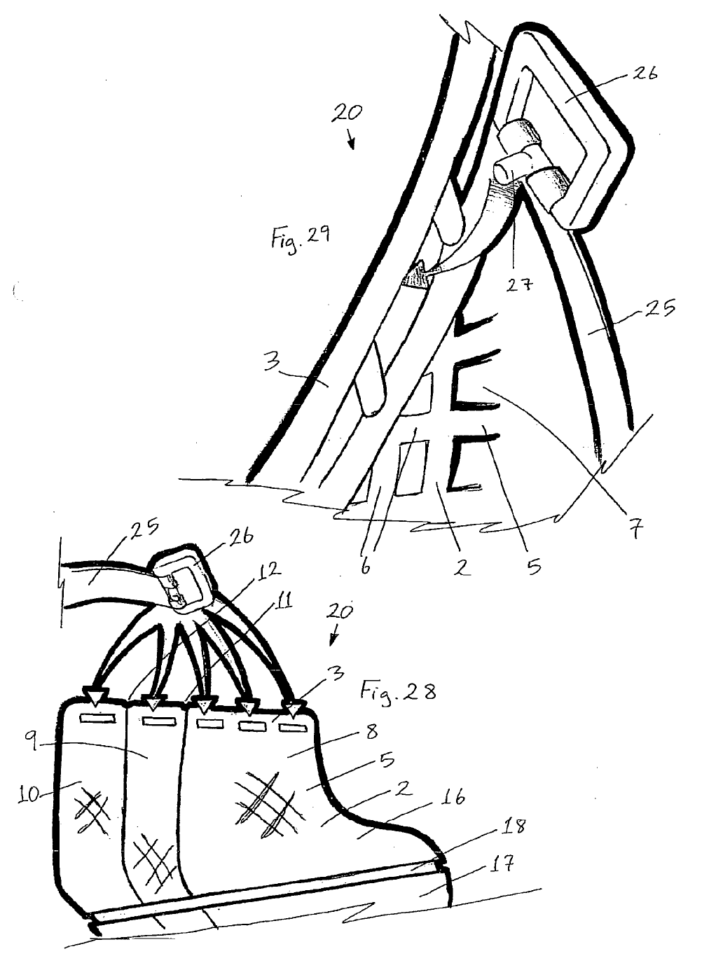

FIGS. 28 and 29 are schematic illustrations of the urging of FIG. 27, in use;

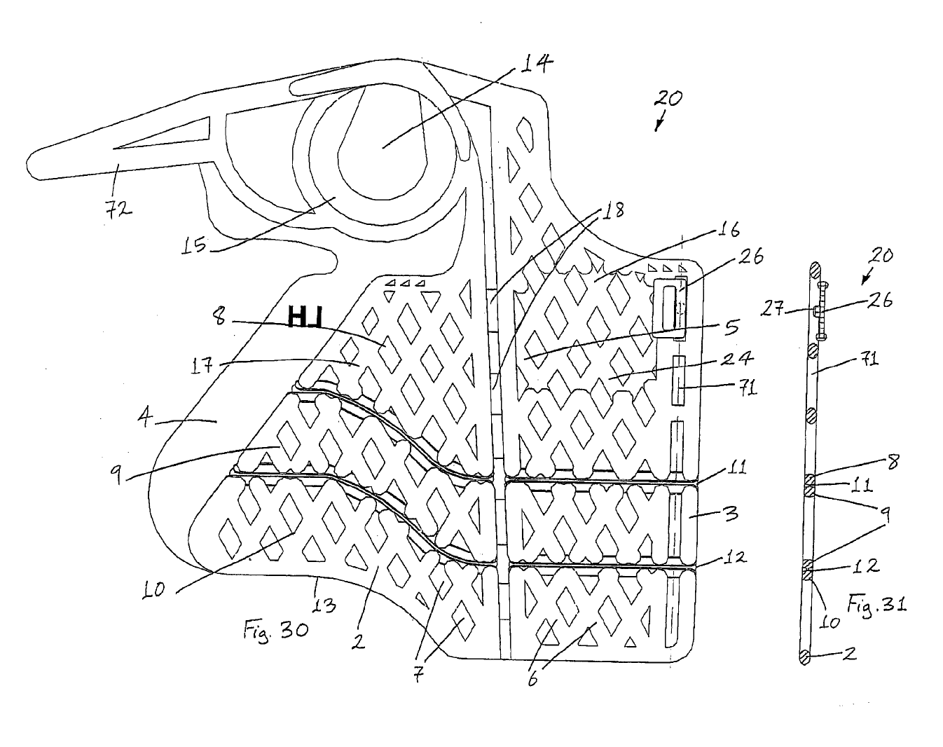

FIG. 30 is plan view of the splint device of FIGS. 2 to 5;

FIG. 31 is a view along line A-A in FIG. 30 and illustrates the buckle section showing that the lug of the buckle does not protrude through the cast material, and that the overall profile is not affected to a great extent;

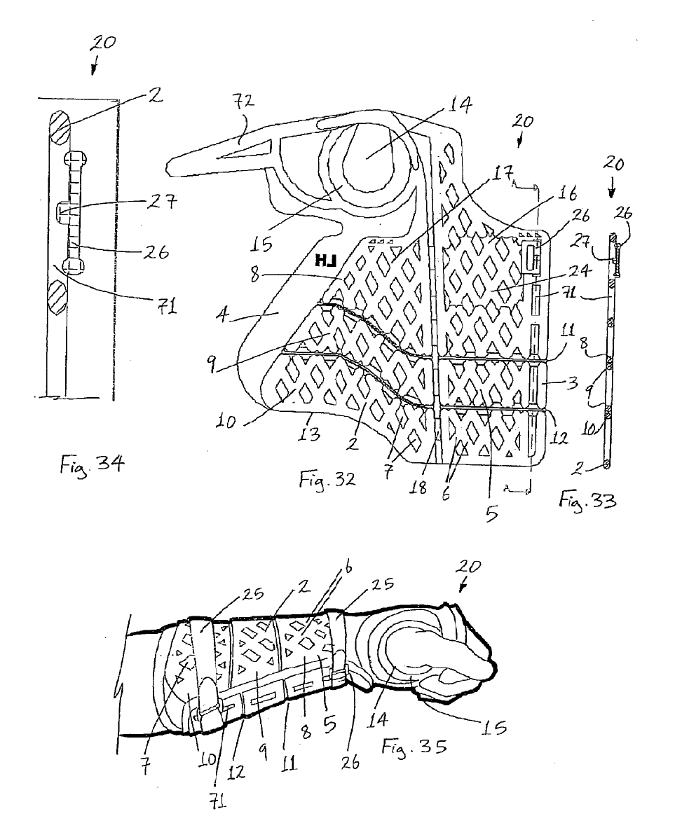

FIG. 32 is a plan view of the splint device of FIGS. 2 to 5;

FIG. 33 is a view along line A-A in FIG. 32;

FIG. 34 is an enlarged view of a part of the splint device of FIG. 33;

FIG. 35 is an isometric view of the splint device of FIG. 32, in use;

FIG. 36 is a plan view of the splint device of FIG. 2 to 5;

FIG. 37 is a view along line A-A in FIG. 36;

FIG. 38 is a view along line B-B in FIG. 36;

FIG. 39 is an enlarged view of a part of the splint device of FIG. 37;

FIG. 40 is an enlarged view of a part of the splint device of FIG. 38;

FIG. 41 to 43 are isometric views of the splint device of FIG. 36, in use;

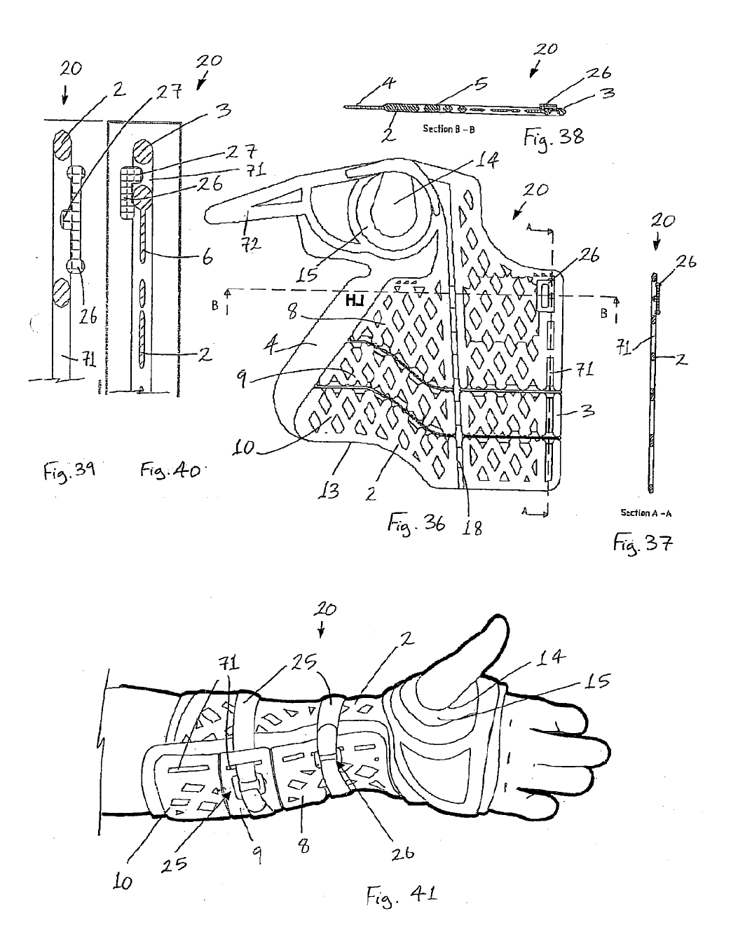

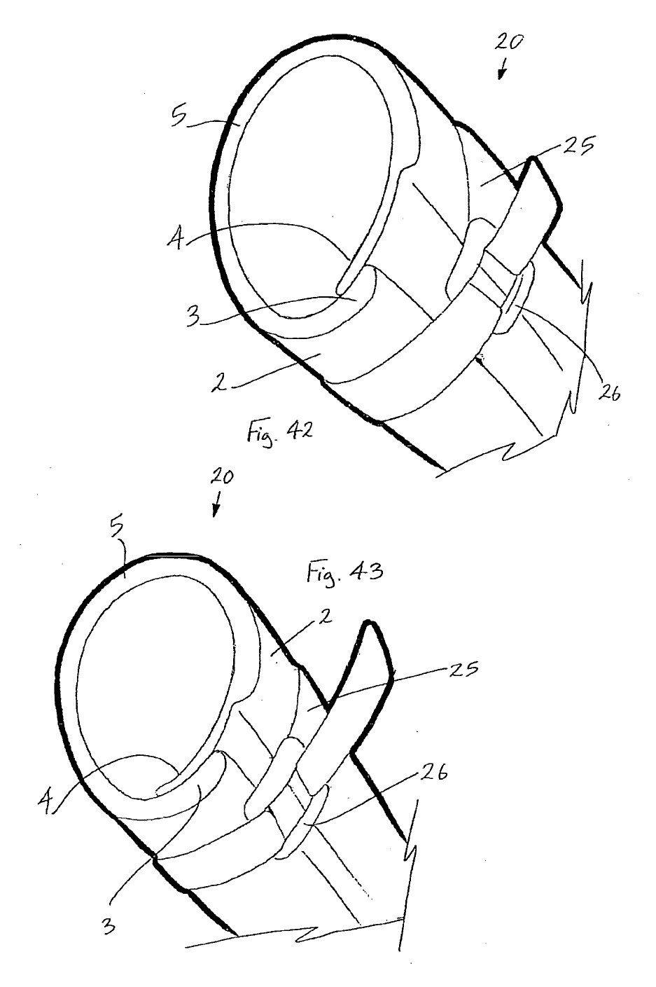

FIGS. 36 to 41 show the features of the splint device in greater detail incorporating an extra section through the cast. This section B-B shows the functionality of the lug of the buckle to anchor the d-loop device into the slots of the end of the cast. FIG. 41 shows that the straps can be positioned through the slots at the end of the cast. In this case, a buckle with a lug may not be required, or the lug may sit in one of the crevices of the hatched mesh geometry. The entire cast is shown with the straps tightening the device. FIGS. 42 and 43 show the functionality of the straps in adding volumetric sizing control by tightening of the straps

FIG. 44 is a plan view of the splint device of FIGS. 2 to 5;

FIG. 45 is a view along line A-A in FIG. 44;

FIG. 46 is an enlarged view of a part of the splint device of FIG. 45;

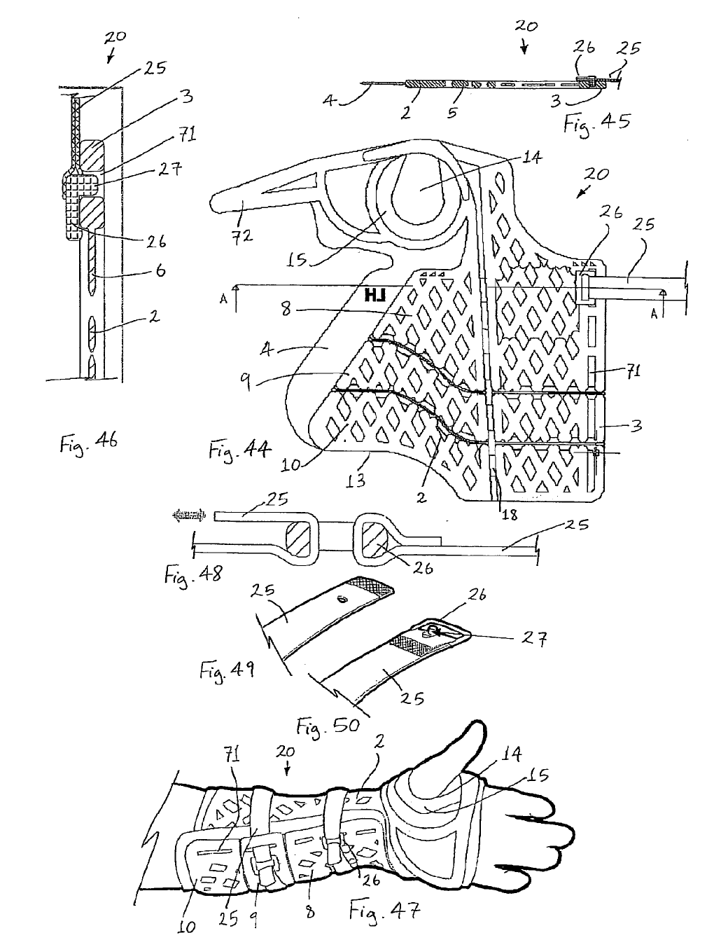

FIG. 47 is an isometric view of the splint device of FIG. 44;

FIG. 48 is a cross-sectional, side view of a part of the urging means of the splint device of FIG. 44;

FIGS. 49 and 50 are isometric views of parts of the urging means of FIG. 48;

FIG. 51 is an isometric view of another splint device according to the invention and shows in more detail the thinned section of the device which is located at one side of the device. This thinned section is tapered.

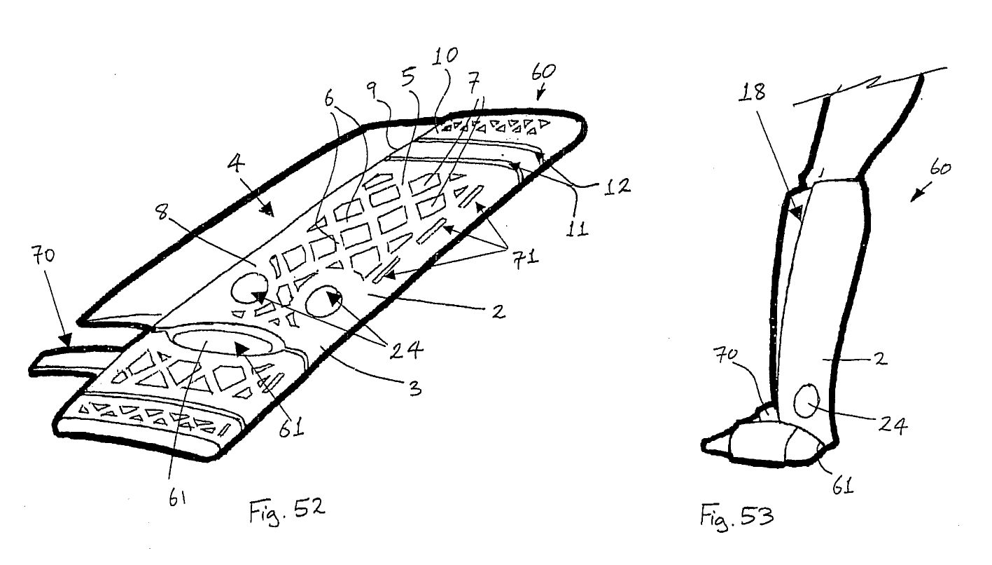

FIG. 52 is an isometric view of a further embodiment of the present invention where the main features of the distal forearm cast shown in FIGS. 1-51 are incorporated into a leg cast.

FIG. 53 is an isometric view of the splint device of FIG. 52, in use, and

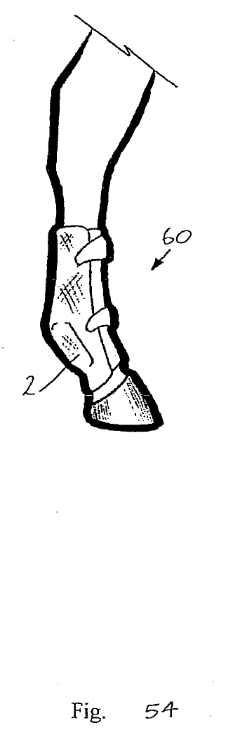

FIG. 54 shows another embodiment of the device using the features described, a similar type device can be used for the treatment of equine injuries and fractures.

FIG. 55 is a schematic view of a device in accordance with the present invention showing the tabs.

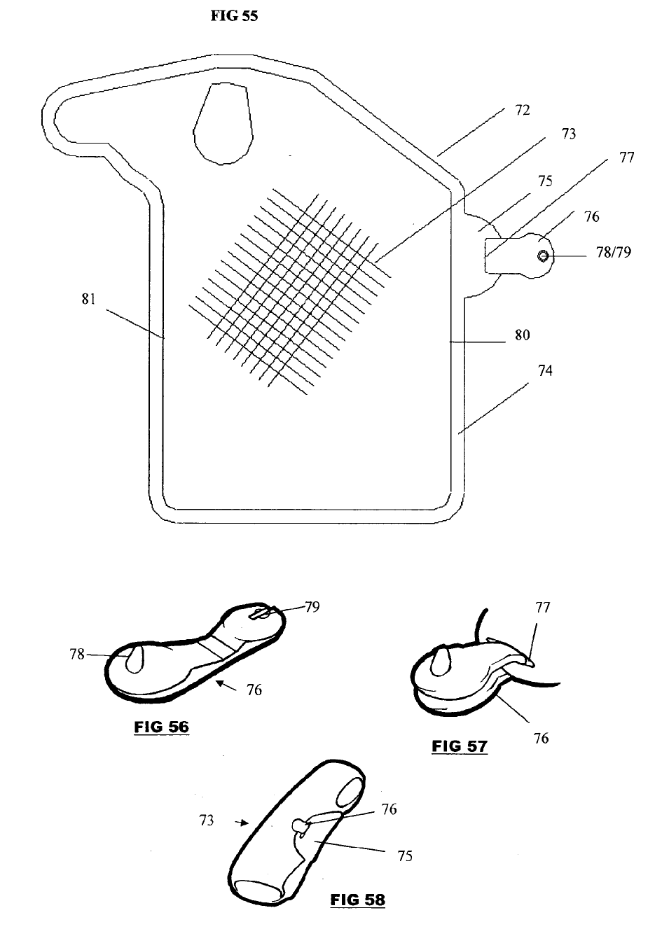

FIGS. 56 and 57 are schematic views of the tab

FIG. 58 illustrates the use of the tab as an indicator of the device integrity.

Referring to the drawings, and initially to FIG. 1 thereof, there is illustrated a distal forearm medical splint device 1 according to the invention. The device 1 comprises a splint member 2 for splinting an object, such as a patient's distal forearm after fracture of a bone of the forearm.

The material of the splint member 2 comprises a low-melt polymer and a ligno-cellulose additive material. In this case the low-melt polymer comprises a thermoplastic, such as polycaprolactone. In this case the lingo-cellulose additive material comprises wood. The wood is provided in the form of a plurality of fibres dispersed throughout the polycaprolactone.

The device 1 may be manufactured using a variety of plastic processing technologies, such as injection moulding, compression moulding, casting, extrusion, and die cutting.

The thermoplastic cast material is radio translucent to aid x-ray examination of the limb without having to remove the device 1.

The low melt temperature polymeric material may comprise a polycaprolactone. The additive material may comprise wood. The additive material may be provided in the form of a plurality of fibres, spheres or platelets. These fibres, spheres or platelets may be dispersed throughout the low melt polymeric material.

The splint member 2 is substantially rigid at ambient temperature, and is formable at a forming temperature above ambient, in this case above about 57° C. By heating the splint member 2 above 57° C., the splint member 2 may be formed into the desired shape and wrapped around the distal forearm to splint the distal forearm.

The cast material is biodegradable and recyclable and has a low activation temperature.

Addition of additives, such as carbon black which may change the thermal characteristics of the cast material, may improve the activation time. Additives such as carbon black have an absorbency of ca. 880 nm which is in the near-infrared. Utilisation of absorption energies of additives incorporated in the matrix material may be used in order to optimise the activation efficiency of the device using a variety of activation means such as IR heaters, convection means such as ovens, conduction means such as water heating, or by radiation means such as microwave gamma or e-beam.

The splint member 2 is provided in a flat form prior to forming. Subsequent to forming around the distal forearm, the splint member 2 is substantially cylindrical. In particular subsequent to forming, the splint member 2 defines a substantially cylindrical interior volume, within which the distal forearm is located.

The interior volume of the splint member 2 may be altered from a first treatment configuration to a second treatment configuration. The interior volume in the first treatment configuration is greater than the interior volume in the second treatment configuration. When the splint member 2 is in the first treatment configuration positioned around the distal forearm, the splint member 2 is suitable for primary treatment of the distal forearm during the primary treatment stage following fracture of the bone, for example for the first 2 to 3 days following the fracture. When the splint member 2 is in the second treatment configuration positioned around the distal forearm, the splint member 2 is suitable for secondary treatment of the distal forearm during the secondary treatment stage subsequent to the primary treatment stage, for example for the subsequent 2 to 3 weeks.

The material of the splint member 2 biases the splint member 2 towards the first treatment configuration.

In one embodiment the invention discloses a single cast designed to function both as a primary and as a secondary treatment for limb fractures and injuries, such as to the distal forearm area.

The volumetric adjustability of the invention allows the cast to serve as both a primary and secondary treatment device for limb fractures and injuries. This is accomplished by the incorporation of the features as described below.

The adjustment of the cast allows for secure support of the injury throughout every stage of primary and secondary treatment to allow for correct bone alignment, healing of damaged or torn tendons or tissue injuries.

The splint member 2 comprises a first side region 3, a second side region 4 on an opposite side of the splint member 2 to the first side region 3, and an intermediate region 5 between the first side region 3 and the second side region 4. The thickness of the second side region 4 is less than the thickness of the intermediate region 5, and the thickness of the second side region 4 is less than the thickness of the first side region 3. The thickness of the splint member 2 varies in a step-wise manner from the intermediate region 5 to the second side region 4. The thickness of the first side region 3 is substantially equal to the thickness of the intermediate region 5.

The first side region 3 and the intermediate region 5 are provided in the form of a mesh of splint elements 6, with a plurality of openings 7 through the splint member 2. The second side region 4 is provided in the form of a continuous body of material.

When the splint member 2 is positioned around the distal forearm, the first side region 3 overlaps over the second side region 4.

The thinned section 4 along one side of the cast allows the corresponding side to overlap without increasing the height of the cast. This thinned area also allows for an overlap to facilitate sizing of small to large diameter limbs.

The volumetrically adjustable splint, by virtue of the ease of adjustability of the cast, reduces the risk of compartmental syndrome. The thinned section 4 of the cast also aids the prevention of compartmental syndrome by providing minimal step thereby allowing for flexibility in adjustability. This minimal step will minimise the cross-over of the ends of the cast thereby eliminating the risk of pinching of the skin and for a uniform surface to circumferentially encase the injured or fractured limb.

The splint member 2 comprises a thumb opening 14 through which the thumb may extend, when the splint member 2 is positioned around the distal forearm. Adjacent to the thumb opening 14, the splint member 2 comprises a continuous body of material 15. When the splint member 2 is heated to the forming temperature, the body of material 15 is movable, for example by rolling the body of material 15 back, to selectively adjust the size of the thumb opening 14. In this manner the size of the thumb opening 14 may be altered to suit the size of the thumb.

With the splint device 1 of the invention, it is not necessary for a clinician to be skilled in wrapping techniques to apply the splint device 1. With the splint device 1 of the invention, folds may be avoided. The roll back area 15 around the thumb allows the cast to be self-locating and custom formed and therefore provide a comfortable and secure fit.

The splint member 2 comprises a main body section 8, a first proximal auxiliary section 9 coupled to the main body section 8, and a second proximal auxiliary section 10 coupled to the first proximal auxiliary section 9. A first connecting section 11 is provided between the main body section 8 and the first proximal auxiliary section 9, and a second connecting section 12 is provided between the first proximal auxiliary section 9 and the second proximal auxiliary section 10.

The thickness of the main body section 8 is substantially equal to the thickness of the first proximal auxiliary section 9, and the thickness of the first proximal auxiliary section 9 is substantially equal to the thickness of the second proximal auxiliary section 10. The thickness of the first connecting section 11 is less than the thickness of the main body section 8 and the first proximal auxiliary section 9. The thickness of the second connecting section 12 is less than the thickness of the first proximal auxiliary section 9 and the second proximal auxiliary section 10.

The main body section 8, the first proximal auxiliary section 9, the second proximal auxiliary section 10, the first connection section 11, and the second connecting section 12 are all formed integrally.

The second proximal auxiliary section 10 may be selectively decoupled from the first proximal auxiliary section 9 by cutting along the second connecting section. 12 with a suitable cutting instrument. Similarly the first proximal auxiliary section 9 may be selectively decoupled from the main body section 8 by cutting along the first connecting section 11. In this manner the longitudinal dimension of the splint member 2 may be altered to suit the size of the distal forearm being splinted.

It will be appreciated that any suitable number of auxiliary sections and connecting sections may be provided to enable the splint member 2 to be used to splint a variety of sizes of distal forearm.

Trimmable sections 9, 10 at a variety of locations are present to make the cast adjustable to accommodate main population sizes. The trimmable sections 9, 10 are designed such that there are no sharp edges in direct contact with the limb. It would be difficult to develop a device for treating limb injuries that would fit the entire population based on valid anthropometric data using a single sized product. By the incorporation of the trimmable sections 9, 10 and volumetric adjustability in the present invention, and utilising the stretchability of the cast material and padding material, it is possible to create a “one-size-fits all” device.

Part of the peripheral edge 13 of the second proximal auxiliary section 10 is curved in a concave form. When the splint member 2 is positioned around the distal forearm, the curved edge 13 fits around the inner side of the elbow.

The half moon area 13 at the base of the cast 1 allows maximum comfort while placed in a sling.

The splint member 2 comprises a first part 16, a second part 17, and a plurality of hinge elements 18 between the first part 16 and the second part 17.

The hinge elements 18 are spaced apart from one another. The hinge axis of each hinge element 18 is in-line with one another.

The hinge elements 18 are formed integrally with the first part 16 and with the second part 17. The thickness of each hinge element 18 is less than the thickness of the first part 16 and less than the thickness of the second part 17.

When the splint member 2 is at ambient temperature subsequent to forming, the hinge elements 18 facilitate hinging movement of the first part 16 relative to the second part 17. In this manner it is possible for the splint member 2 to be opened up to remove the distal forearm from the splint member 2.

Tabs located at one end of the device 1 facilitate easy removal of the cast upon breaking of the tab along the hinge. This may also be used as an indicator of unauthorised removal. Easy removal of the device means that use of a cast saw or cutting device is not required.

The volumetrically adjustable tab around the palm area allows for adjustment at the crease of the palm of the hand while retaining restriction of the movement of the hand.

FIG. 1 illustrates the extended radial bar 70, the strap retainers 71, the hinge area 18, the trimming lines 11, 12, the thinned thumb area 15, the thinned area-overlap 4, and the half-moon recess 13.

In use, the splint member 2 is heated from ambient temperature to above 57° C. The splint member 2 is formed into the desired shape and wrapped around the distal forearm in the first treatment configuration. The first side region 3 is overlapped over the second side region 4, and the thumb is extended through the thumb opening 14. If necessary the size of the thumb opening 14 is adjusted.

If necessary the second proximal auxiliary section 10 is decoupled from the first proximal auxiliary section 9, and if necessary the first proximal auxiliary section 9 is decoupled from the main body section 8.

After the primary treatment stage, the splint member 2 is moved from the first treatment configuration to the second treatment configuration.

After the second treatment stage, the splint member 2 is opened up by hinging the first part 16 relative to the second part 17. The distal forearm is then removed from the splint member 2.

The volumetrically adjustable device 1 may cover 5-95 percentile of arm sizes by incorporating stretchable and trimmable sections. This is achieved using the half moon section 13 which broadens the sizing range. The device 1 may be easily adjusted for increases or decreases in swelling.

The volumetrically adjustable splint 1 may reduce the cost to the hospital as there is no need for stockinette or extra padding. Reduced application time may result as once the preformed/unformed product is activated, simple adjustment of the strapping may be employed to change the volume being encased by the cast. The volumetric adjustability of the device enables compensation of swelling which occurs at various stages of the duration of the required treatment. Removal or replacement of the device between primary and secondary treatment is not necessary, which reduces potential risk and discomfort. It is advantageous to the clinic, hospital and user that the device may be used to treat a variety of injuries and fractures in its entirety.

In FIGS. 2 to 12, 14 to 22, and 27 to 50, there is illustrated another splint device 20 according to the invention, which is similar to the splint device 1 of FIG. 1, and similar elements in FIGS. 2 to 12, 14 to 22, and 27 to 50 are assigned the same reference numerals.

In this case the splint device 20 comprises a spacer member 21 attached to the splint member 2. When the splint device 20 is positioned around the distal forearm, the spacer member 21 is located between the splint member 2 and the distal forearm.

The spacer member 21 comprises a foam, padding bandage of a hydrophilic material. The spacer member 21 is of a low elastic recovery material. The force required to linearly extend a 30 mm wide by 100 mm gauge length sample of the spacer member 21 by 40% of its original length is less than or equal to 2.5N, and is preferably approximately 1.2N.

In one case the spacer member 21 comprises a polyester fibre in combination with 5% to 15% elastene fibre.

The padding material 21 is very stretchable with limited recoil memory. 40% strain can be achieved using a force of between 1.2 and 2.5 N.

The measurements for the low elastic recovery/stretchability of the padding 21 are as follows:
for linear extension within the elastic region, a maximum of 2.5N to reach 40%, but preferably 1.2N to reach 40% strain.

The measurements for the low elastic recovery of the padding 21 are for a specific fabric that is used for the splint device 20. Measurements were carried out on a 30 mm wide by 100 mm gauge length sample with a thickness of 2.3 mm.

One side or surface of the padding 21 may be treated or the entire padding 21 may be completely impregnated with a hydrophilic coating such as Poly Vinyl Pyrrolidinone (PVP) or poly acrylic acid to improve absorption/wicking properties.

Alternatively one side or surface of the padding 21 may be treated or the entire padding 21 may be completely impregnated with a hydrophobic coating such as PTFE (Polytetrafluoroethylene) to repel moisture and inhibit absorption.

Alternatively, the padding may be treated with a hydrophilic coating on one side and a hydrophobic coating on the other. Such an arrangement would absorb moisture at the skin and repel water at the surface.

Silicone may be applied by a dipping process. This process may impart a hydrophobic coating on the padding material 21. A similar process using a low concentration may reduce the hydrophobicity of the coating.

Since the padding material 21 is adhered to the cast, application time may be reduced since there is no requirement for further support or padding of the injured area.

In a further embodiment, the padding material 21 has a hydrophilic layer with wicking properties. The wicking properties of the padding material may reduce the occurrence of skin maceration.

The padding material 21 may have specific material properties in which the wicking properties reduce the moisture contact on the skin. This may be achieved by capillary action or by means of a hydrophilic coating on the padding 21 in contact with the skin.

The padding material 21 may have a hydrophobic coating to repel moisture on the surface with adequate aeration in order to allow for air circulation around the covered skin area in order to reduce moisture—skin contact.

The hydrophilic coating applied to the padding 21 may be a function of the padding material. A number of padding materials with a hydrophilic coating that can aid in the wicking properties of the padding material may be employed with the splint device of the invention.

The wicking function of the padding 21 may be enhanced by the open latticed structure of the outer splint 2. The three dimensional structure of the padding 21 and the orientation of the fibres in the padding 21 transmit moisture, including sweat, from the whole surface area in contact with the skin directly and indirectly through the reduced area at the apertures in the open latticed structure of the outer splint 2. On reaching the outer surface of the padding 21 in the apertured regions of the splint 2, the moisture is dissipated by evaporation. Examples of suitable materials include material D3 from Gehring Textiles (USA), Rosidal Soft from Lohmann & Rauscher; or Drylyte from HIND.

In a further embodiment, the padding material 21 may support the cast once activated and in a pliable state, and may be stretchable to reach the required sizes of the 95 percentile of the population.

The open surface of the cast design facilitates rapid drying of the padding material 21 particularly when a breathable wicking padding is used. The underlying padding 21 may be stretchable and ideally have a low elastic recovery. The underlying padding 21 may act as a support to the activated cast during forming.

Part of the first connecting section 11 is curved in a concave form and part of the second connecting section 12 is curved in a concave form in a similar manner to the peripheral edge 13 of the second proximal auxiliary section 10.

FIGS. 6 to 8 illustrate the thermoplastic universal mouldable device with volumetric adjustability formed around the distal forearm of either the right hand side (RHS) or the left hand side (LHS).

FIGS. 10 and 11 illustrate the trimmed detail.

FIGS. 6 to 8 illustrate the formed device on hand both LHS and RHS.

FIG. 2 illustrates the roll back area around thumb 15, the hinge section 18 along the centre of the cast, and the trimmable sections 11, 12.

FIG. 3 illustrates the tamper proof tab 70, the volumetric hand adjustable tab 72, and the half moon areas 11, 12, 13.

FIG. 4 illustrates the bony prominence recess 24, the slots 71 positioned to facilitate positioning of the straps 25, and the thinned section 4 along the side of the cast.

FIG. 5 illustrates the padding 21.

FIG. 8 illustrates the device shaped on the lower distal arm.

As illustrated in FIGS. 14 and 15, the splint member 2 has a flat outer surface 22 for facing away from the distal forearm being splinted, and a flat inner surface 23 for facing towards the distal forearm being splinted. In this manner the splint member 2 may be used to splint a left distal forearm or a right distal forearm, as illustrated in FIGS. 16 and 17.

FIGS. 16 and 17 show the unformed device sitting on either the right or the left hand. This shows the universal applicability of the device.

FIG. 15 illustrates the section of cast showing flat top and bottom.

FIGS. 16 and 17 illustrate the unformed device showing versatility of application to both left and right hand.

FIGS. 14 and 15 illustrate the end of the device showing the flat top and bottom sides 22, 23 of the device. Also shown are the slots into which the D-Loop device can fit.

FIGS. 18 to 21 illustrate the exploded trimming section. FIG. 20 illustrates the trim section detail uncut, and FIG. 21 illustrates the trim section detail cut.

As illustrated in FIG. 22, the intermediate region 5 comprises a localised section 24 of reduced thickness. The section 24 is suitable for positioning around a protruding part of the distal forearm, or a bony prominence of the distal forearm. Because of the reduced thickness, the section 24 has greater flexibility than the remainder of the intermediate region 5 when the splint member 2 is at the forming temperature.

The bony prominence recess pressure relief area 24 prevents excessive pressure points at the bony prominence. An example of this is an ulna recess area on a distal forearm cast or an ankle, tibular or fibular, recess area on a leg cast.

FIG. 22 illustrates the bony prominence recess 24.

The splint device 20 also comprises two straps 25. When the splint member 2 is positioned around the distal forearm, the straps 25 are wrapped around the splint member 2 to urge the splint member 2 towards the second treatment configuration.

A fastener element is provided at one end of each strap 25 to enable the straps 25 to be retained in position wrapped around the splint member 2. It will be appreciated that a variety of different types of fastener elements may be employed, for example a hook and loop fastener such as that available under the trade name Velcro.

The buckle 26 of the strap 25 comprises a protruding male element 27 which may be inserted into one of the splint member openings 7 (FIG. 29). In this manner the strap 25 may be coupled to the splint member 2 to enable the strap 25 to be retained in position wrapped around the splint member 2.

FIG. 31 illustrates the D-loop with the lug 27 fitting into the end of the device.

FIG. 27 illustrates the buckle 26 of the D-loop with the bar lug 27.

FIGS. 27 to 29 are 3-D schematic drawings of a D-loop used as a mechanism for tightening the strap 25 that is used for the volumetric adjustability of the cast. Also located on this D-loop is the protrudence “lug” 27 which is used to fix or secure into the slots 7 shown in FIG. 4.

FIGS. 30 and 31 illustrate the D-Loop 26 placed in the groove 7 of the device. The D-Loop device has one lug 27 that sits snug into the side of the cast.

FIG. 34 illustrates the detail of the buckle section 26.

FIG. 50 illustrates the lug 27 on the buckle 26.

The strap 25 may be inserted through one of the splint member openings 7 (FIG. 41). In this manner the strap 25 may be coupled to the splint member 2 to enable the strap 25 to be retained in position wrapped around the splint member 2.

Ports or slots 7 in the cast allow for the positioning of the retention or adjustable straps 25 thereby preventing them from slipping out of place. These straps 25 facilitate easy cast adjustment on reduction of swelling of the injured limb. The straps 25 may also use the slots 7 to be weaved through in order to facilitate accurate and permanent placement, if required. The straps 25 may be located between the slots 7 and the padding material 21.

The straps 25 may have a buckle 26 which may comprise a D-Loop or concave buckle at the end to aid improved adjustment of the cast.

One embodiment of such a D-loop is for the D-loop to include a modified buckle. In this embodiment, sections of the loop will protrude into sections of the cast and thus prevent slippage of the straps 25.

FIG. 31 illustrates the buckle section 26 showing that the lug 27 of the buckle 26 does not protrude through the cast material, and that the overall profile is not affected to a great extent.

FIGS. 36 to 41 show the features of the splint device in greater detail incorporating an extra section through the cast. This section B-B shows the functionality of the lug 27 of the buckle 26 to anchor the D-loop device into the slots 7 of the end of the cast. FIG. 41 shows that the straps 25 may be positioned through the slots 7 at the end of the cast. In this case, a buckle 26 with a lug 27 may not be required, or the lug 27 may sit in one of the crevices of the hatched mesh geometry. The entire cast is shown with the straps 25 tightening the device.

FIGS. 42 and 43 show the functionality of the straps 25 in adding volumetric sizing control by tightening of the straps 25.

FIG. 41 illustrates the strap 25 through the slot 7 and the buckle 26 with the lug 27 in the slot 7.

The thickness of the splint member 2 may vary in a gradual manner from the intermediate region 5 to the second side region 4, as illustrated in FIG. 13.

A thinned section 4 along the side of the device allows the cast to completely encircle the limb without inhibiting swelling and with no danger of pinch points. This thinned section 4 may be a tapered cross-section of one of the ends of the device, as shown in FIG. 13.

FIG. 13 illustrates the thinned section showing the tapered region.

In FIGS. 10 to 13 the thinned flash section 4 of the device is shown. The thinned section 4 can be tapered, and depending on the mode of manufacture, can be flat on one side, or evenly tapered towards a thinned midsection.

FIGS. 23 and 24 illustrate a further splint device 30 according to the invention, which is similar to the splint device 1 of FIG. 1, and similar elements in FIGS. 23 and 24 are assigned the same reference numerals.

In this case the first part 16 is movable in a hinging manner relative to the second part 17.

The first side region 3 does not overlap the second side region 4. The thickness of the first side region 3 is substantially equal to the thickness of the second side region 4.

Referring to FIGS. 25 and 26 there is illustrated another splint device 40 according to the invention, which is similar to the splint device 30 of FIGS. 23 and 24, and similar elements in FIGS. 25 and 26 are assigned the same reference numerals.

In this case the splint member 2 comprises the first part 16, the second part 17 and a third part 41. A plurality of hinge elements 42 are provided between the second part 17 and the third part 41. The second part 17 is movable in a hinging manner relative to the third part 41.

The hinged section along the cast facilitates volumetric adjustment for swelling and easy removal. There can exist a number of hinged sections, for example one to six hinged sections along the length or part of the length of the device. These hinged sections improve the flexibility of the device to aid the volumetric adjustability of the device, and also to allow for ease of removal of the device without compromising further the injury of the limb. A preferred embodiment of the device has two hinged sections or three hinged sections. Examples of these hinged sections are shown in FIGS. 23 to 26.

FIGS. 23 to 26 illustrate the hinged sections of the device.

FIGS. 23 and 24 illustrate the open hinge on the device.

FIGS. 25 and 26 illustrate the open double hinge on the device.

In FIG. 51 there is illustrated another splint device 50 according to the invention, which is similar to the splint device 1 of FIG. 1, and similar elements in FIG. 51 are assigned the same reference numerals.

FIGS. 52 and 53 illustrate a further splint device 60 according to the invention, which is similar to the splint device 1 of FIG. 1, and similar elements in FIGS. 52 and 53 are assigned the same reference numerals.

In this case the splint device 60 is a leg medical splint device. The splint member 2 may be employed to splint a patient's leg after fracture of a bone of the leg.

The splint member 2 comprises a heel opening 61 through which the heel may extend, when the splint member 2 is positioned around the leg.

FIGS. 52 and 53 illustrate the leg splint.

FIGS. 52 and 53 illustrate an example of the device for use as a leg cast. FIG. 52 illustrates the thinned section 4 along the side of the cast, the volumetric adjustable tab 70, the hole 14, the roll back areas or bony prominences 24, the straps 71, and the trim sections 11, 12.

FIG. 53 illustrates the possible hinge section 18.

It will be appreciated that the leg splint device 60 of the invention is suitable for use with a human leg (FIGS. 52 and 53), or with an animal leg such as a horse's leg (FIG. 54).

The device 60 is not limited to use on human injured or fractured limbs. The features described in this orthopaedic device 60 may be used on animals as shown in FIG. 54 where the device 60 is used for treatment of an equine injury or fracture.

Referring to FIG. 55, device 72 consists the first member in the form of a thermoplastic sheet 73 having a web-like configuration to allow it to extend in one or two dimensions when warmed to its plastic state. An underlying fabric sheet 74 comprises the second member and is bonded to the lower surface of sheet 73. Although the fabric sheet 74 extends generally beyond the edges of sheet 73, one area further projects to form a tongue 75. A slit 77 is formed in the fabric at this region, through which passes tab 76.

As shown in FIG. 56, tab 76 consists of a sheet of material, typically of a dumbbell shape. An upstanding peg 78 is formed in one end of the tab, which co-operates with a hole 79, formed in the other end of the tab. FIG. 57 depicts the formed tab in which one end is passed through the slit 77 and the tab material bent about its mid-line until peg 78 is aligned with and passed through hole 79. The end of the peg is then deformed thereby preventing movement of the peg out of registry with hole 79.

As shown in FIG. 58, the device is heated, e.g. in a water bath, until it softens and becomes flexible. The device, in this state is then applied to a body part e.g. an arm and shaped around the part to form a generally tubular splint with the opposed edges 80, 81 (FIG. 55) adjacent each other. Edge 80 together with tongue 75 and tab 76 are extended over edge 81 and tab 76 pressed into the thermoplastic mass of sheet 73. On cooling of the device, tab 76 forms a unitary and inseparable part of sheet 73.

During application of the device, the fabric holding the tab is stretched to position and the tab, which is malleable when heated, is adhered to the body of the device. Although the cast is sealed the excess fabric between the cast and tab provides the required stretch for the cast to adjust volumetrically to allow for swelling.

To remove the device the fabric between the tab and the cast is cut. The cast can then be readily opened for easy removal. If excessive force is applied to the fabric between the tab and cast through unauthorised removal the slit 77 elongates and eventually the fabric will rip/tear providing an indication that the cast was either removed or tampered with.

The manufacturing process for obtaining a black colouring of the splint material is as follows:

Black colour may be obtained by incorporating a loading of between 0.01 wt % and 0.001 wt % carbon black. This loading preferably will be between 0.008 wt % and 0.004 wt % carbon black.

The carbon black may be dispersed in a carrier which is compatible with the polycaprolactone matrix material. Typical carriers may include, but are not limited to oils, such as olive oil or linseed oil.

The term “carbon black” is well known to those skilled in the art of colouring and modifying plastic materials (similar to soot but has much larger surface area to volume ratio).

The pigment can be hand-blended, or blended using a mixing machine, or blended using a secondary hopper during extrusion. Blending can take place at room temperature or during processing of the blend at temperatures of between 70° C. and 132° C.

The manufacturing process for obtaining a particular chemical smell of the splint material is as follows:

The fragrance employed in the invention should be compatible with the polymer that is employed. Some polymer and fragrances are not compatible with each other. That means that a particular fragrance can not be delivered with a specific polymer.

Many types of fragrances can be employed in the present invention, the only limitation being the compatibility with the polymer matrix being employed. Suitable fragrances include but are not limited to fruits such as almond, apple, cherry, grape, pear, pineapple, orange, strawberry, raspberry; musk, flower scents such as lavender-like, rose-like, iris-like, carnation-like, herbal scents, woodland scents derived from pine, spruce and other forest smells. Fragrances may also be derived from various oils, such as essential oils, or from plant materials such as peppermint, spearmint and the like.

The fragrance can be added to the polymer mix directly prior to compounding. Alternatively, well know materials such as surfactants, emulsifiers, polymers to encapsulate the fragrance can also be employed and added to the compounding recipe. Alternatively, the fragrance can be directly added to the padding material. Alternatively, the fragrance can be located in the packing material of the device, from which the device can absorb the fragrance during storage.

In one embodiment the invention may provide an orthopaedic cast or splint for applications such as primary and secondary treatment of a distal forearm injury or fracture. In one embodiment the invention provides for volumetric adjustability of the cast by using features such as the cast hinge, the thinned end section, the slots and retention straps, and stretchability of the activated cast using the padding material as a support mechanism. In one embodiment the device may be manufactured using a variety of plastic processing technologies such as injection moulding, compression moulding, casting, extrusion, and die cutting.

In its various embodiments the invention thus provides
An orthopaedic cast which does not deform significantly in the pliable state during application. The pliable cast may be supported from deformation by virtue of being adhered to the padding material. In one embodiment the invention further provides an orthopaedic device having wide applicability and a unique combination of desirable properties including volumetric adjustability, which may be used for treatment of primary and secondary injuries in-situ, without removal or re-moulding of the device.
An orthopaedic device, orthopaedic materials and methods for treating primary and secondary lower forearm injuries or other limb injuries, such as distal leg: fractures or humeral injuries. The invention may provide improved methods for immobilisation, bracing, casting, protection and support of limbs and body parts of humans and animals. The present invention may be termed a cast, a splint, a brace, a protective support, or a device. Examples of the field of use of this invention include, but are not limited to, the treatment of lower arm injuries, such as distal radius or ulna fractures, scaphoid fractures, metacarpal and other kind of fractures or injury such as soft tissue injuries, and can be used for primary and secondary casting. Also included in the field of use of this invention is any limb that has been injured or fractured, including humeral injuries, leg injuries and the like.
A volumetric adjustable orthopaedic cast that can be used for treatment of primary and secondary fractures and injuries of limbs, such as the distal forearm.
The ability to negate the use of gloves or speciality handling of the activated cast since the padding material acts as an insulating layer and the material is activated between temperatures of 45 and 70° C. The padding material may not be affected by the activation temperature. Activation of the device can mean that external energy is applied to the device which in its inactivated form may be rigid. After activation, the device may be pliable and can be manipulated to conform to any size or contour of the injured limb for which the device is designed. On cooling and recrystallisation, the material of the device may revert back to its original rigid and tough non-pliable form.
A material which has an additive that may render it “non-tacky” once activated.
Provides a cast material which may include specific additives that may change the thermal characteristics such that improved activation time may result. Examples of such additives include carbon black or carbon fibres.
Includes features which may be applied to a wide range of orthopaedic uses involving thermoplastic mouldable splints or casts for primary immobilisation of an injured or fractured limb, and subsequent volumetric adjustment for secondary treatment of the injured limb. The stretchability of the activated cast and padding support enables the invention to fit a wide size range of the population.

1-30. (canceled)

31. A custom mouldable orthopaedic device adapted, in use, to be volumetrically variable, including a first member, adapted to be extensible, flexible and conformable around a body part comprising a sheet of thermoplastic material having first and second opposable edge regions, and a second member comprising a layer of an elastically extensible material, bonded to one surface of the first member and adapted to form an interposing layer between the first member and the body part when said first member is adapted to conform around said body part, wherein the elasticity of the material is such that the force required to linearly extend a 100 mm×30 mm×2.3 mm sample, of the material by 40% of its original length is not more than 2.5N and wherein at least a portion of at least one of said edge regions has a thickness less than that of the region intermediate said edge regions, said edge portion being adapted to overlap the opposed edge when said first member is conformed around a body part

32. The device as claimed in claim 31 wherein the material is a breathable spacer fabric

33. The device as claimed in claim 31 wherein the material is a woven, non-woven, knitted or foam material

34. The device as claimed in claim 32 wherein the breathable material has a moisture vapour transmission rate of at least 300 gm/m²/24 hours

35. The device as claimed in claim 34 wherein the material is a hydrophilic material or contains a hydrophilic component

36. The device as claimed in claim 31 wherein the force required to linearly extend a piece of the spacer member, 30 mm wide by 100 mm in length by 2.3 mm thick, by 40% of its original length is about 1.2N.

37. The device as claimed in claim 31 wherein the thickness of the second edge region is less than the thickness of the first edge region.

38. The device as claimed in claim 31 wherein the region of the sheet intermediate said opposed edge regions comprises first and second parts, each part being movable relative to the other.

39. The device as claimed in claim 38 comprising at least one hinge element for providing a hinging movement of the first part relative to the second part generally along a line parallel to said edges.

40. The device as claimed in claim 39comprising a plurality of spaced apart hinge elements.

41. The device as claimed in claim 39 wherein the hinge element is integrally formed with at least one part of said intermediate region.

42. The device as claimed in claim 41wherein said hinge element comprises an intermediate region between said first and second parts, having a thickness less than that of said parts.

43. The device as claimed in claim 39 wherein the hinge element is an external element attached to said intermediate region.

44. The device as claimed in claim 31 wherein at least at least a part of the first member has a plurality of openings extending there through to define a web-like configuration.

45. The device as claimed in claim 31 in which a part of said fabric layer extends beyond said first edge and wherein a tab, adapted to be permanently bonded to said first member, is flexibly attached to said extended part

46. The device as claimed in claim 43 wherein said tab and extended part are arranged to permit the tab to contact the first member in the region of the second edge or an area of the intermediate region adjacent the second edge when said device is conformed around the body part.

47. The device as claimed in claim 31 in which the thermoplastic material comprises a polycaprolactone and an additive selected from ligno-celluloses, carbon, mica, polyanaline, clays or silica gels and wherein the material is formable at temperatures above ambient temperatures and is substantially rigid at ambient temperature.

48. The device as claimed in claim 31 further comprising a strap for securing the device when conformed around a body part.

49. A custom mouldable orthopaedic device adapted, in use, to be volumetrically variable, including
(a) a first member, adapted to be extensible, flexible and conformable around a body part comprising a sheet of thermoplastic material having first and second parts, each having an edge region, at least a portion of at least one of said edge regions has a thickness less than that of the region intermediate said edge regions, said edge portions being adapted to be opposed edge when said first member is conformed around a body part,
(b) at least one hinge element for providing a hinging movement of the first part relative to the second part generally along a line parallel to said edges.
(c) a second member comprising a layer of an elastically extensible breathable material having a moisture vapour transmission rate of at least 300 gm/m²/24 hours, bonded to one surface of the first member and adapted to form an interposing layer between the first member and the body part when said first member is adapted to conform around said body part, wherein the elasticity of the material is such that the force required to linearly extend a 100×30×2.3 mm sample of the material by 40% of its original length is not more than 2.5N and
(d) means for retaining the device in position when conformed around a body part.

50. The device as claimed in claim 49 wherein the means for retaining the device in position when conformed around a body part comprises a strap

51. The device as claimed in claim 49 in which the thermoplastic material comprises a polycaprolactone and an additive selected from ligno-celluloses, carbon, mica, polyanaline, clays or silica gels and wherein the material is formable at temperatures above ambient temperatures and is substantially rigid at ambient temperature.

52. A method for immobilising a body part including the steps of;
(a) heating an orthopaedic device comprising a first member comprising a sheet of thermoplastic material having first and second opposable edge regions, and a second member comprising a layer of an elastically extensible material, bonded to one surface of the first member and adapted to form an interposing layer between the first member and the body part when said first member is adapted to conform around said body part, wherein the elasticity of the material is such that the force required to linearly extend 100×30×2.3 mm sample of the material by 40% of its original length is not more than 2.5N and wherein at least a portion of at least one of said edge regions has a thickness less than that of the region intermediate said edge regions, to render said first member extensible and flexible,
(b) applying and conforming said device around a body portion whereby said edge portions oppose or overlap each other,
(c) allowing the device to set to form a first configuration
(d) subsequently conforming the device to changes in the body part configuration by urging the regions edge to overlap further or less, thereby, changing the total volume of the device.
(e) securing the device to maintain the changed relationship of the edge regions

53. The device as claimed in claim 50 wherein the device is secured by a strap for when conformed around a body part in step (d).
